(19) United States
(12) Patent Application Publication
(10) Pub. No.: US 2026/0183243 A1
(43) Pub. Date: Jul. 2, 2026

(54) PHARMACEUTICAL COMPOSITIONS OF NOR-UDCA

(71) Applicant: SHILPA MEDICARE LIMITED, Raichur (IN)

(72) Inventors: Channabasayya TIPPIMATH, Bengaluru (IN); Hanumantharaja RAMACHANDRAPPA, Bengaluru (IN)

(21) Appl. No.: 19/399,370

(22) Filed: Nov. 24, 2025

Related U.S. Application Data
(63) Continuation of application No. 18/998,287, filed on Jan. 24, 2025.

(30) Foreign Application Priority Data
Jul. 25, 2022 (IN) 202241024145

(51) Int. Cl. A61K 9/20; A61K 31/575

(52) U.S. Cl. CPC A61K 9/2054; A61K 9/2013; A61K 9/2027; A61K 9/2077; A61K 31/575

(57) ABSTRACT

The present invention relates to the pharmaceutical compositions comprising nor-UDCA or pharmaceutically acceptable salt or ester thereof and the process for preparation. The present invention most preferably discloses the solid oral dosage form of nor-UDCA preferably in the form of tablets, capsules, disintegrating tablets and effervescent tablets.

### FIELD OF THE INVENTION

The present invention relates to the pharmaceutical composition comprising 24-nor-ursodeoxycholic acid (nor-UDCA) or pharmaceutically acceptable salt or ester thereof and the process for preparation thereof. The present invention more specifically relates to the pharmaceutical composition comprising at least about 30% w/w of nor-UDCA or pharmaceutically acceptable salt or ester thereof and at least one pharmaceutically acceptable excipient.

### BACKGROUND OF THE INVENTION

24-nor-ursodeoxycholic acid (nor-UDCA) is an ursodeoxycholic analog (bile acid derivative) used for the treatment and/or the prevention of liver diseases, preferably chronic liver diseases. Nor-UDCA has the following structural formula

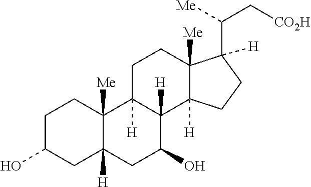

EP U.S. Pat. No. 624,595B1 (“EP '595 Patent”) discloses nor-UDCA, its method for preparation and its use especially to lower cholesterol at a daily dose depending on body weight and constitution of the patient in the range of 3 mg to 5000 mg, preferably in the dose range 10 to 1000 mg. Further EP '595 Patent discloses various dosage forms, preferably oral dosage forms in the form of tablets, capsules or liquids.

U.S. Pat. No. 8,951,995 (“US '995 Patent”) discloses a method of treating an inflammatory cholestatic liver disease in a subject by administering the subject a pharmaceutical composition comprising nor-UDCA and/or pharmaceutically acceptable salt or ester thereof; wherein the inflammatory cholestatic liver disease is primary sclerosing cholangitis (PSC), primary biliary cirrhosis (PBC) or progressive familial intrahepatic cholestasis, in particular progressive familial intrahepatic cholestasis type 1, 2 and 3, cystic fibrosis, drug-induced cholestasis or a noncholestatic liver disease such as chronic viral hepatitis (B,C,D), alcoholic and non-alcoholic steatohepatitis, autoimmune hepatitis, hemochromatosis, Wilson disease and alpha-1-antitrypsin deficiency. Further US '995 Patent discloses solid dosage forms for oral administration that includes tablets, preferably effervescent or chewable tablets, capsules, pills, powders and granules, such solid dosage forms, 24-nor-ursodeoxycholic acid can be admixed with regularly used substances like sucrose, mannitol, sorbitol, starch and starch derivatives, lactose, lubricating agents (e.g. magnesium stearate), disintegrants and buffering agents. Tablets and pills can also be prepared with enteric coatings in order to prevent that 24-nor-ursodeoxycholic acid is affected by the stomach acids and enzymes. As immediate release tablets, these compositions may further comprise microcrystalline cellulose and/or dicalcium phosphate.

U.S. Pat. No. 9,512,167 discloses chemically pure polymorph of nor-UDCA or of a pharmaceutically acceptable salt thereof, wherein the total amount of chemical impurities is less than 0.5%, at least 60% of the polymorph particles have a size <10 μm, and wherein said polymorph contains substantially no detectable amorphous nor-UDCA. Further US '167 Patent discloses solid dosage forms for oral administration that includes tablets, preferably effervescent or chewable tablets, capsules, pills, powders and granules.

The above prior-arts discloses the pharmaceutical compositions for oral administration that include tablet dosage form comprising nor-UDCA. The pharmaceutical tablets comprising nor-UDCA poses certain drawback, from which the problems are dissolution rate and the disintegration of the tablet varies from batch to batch, with some batches having unacceptably low rates.

In view of above disadvantages there exists a need to develop a pharmaceutical composition, most preferably a tablet dosage form comprising nor-UDCA or pharmaceutically acceptable salt or ester thereof and a pharmaceutically acceptable excipient that provides consistent disintegration and dissolution of tablet dosage form from batch to batch.

### SUMMARY OF THE INVENTION

The present invention relates to a pharmaceutical composition comprising 24-nor-ursodeoxycholic acid (nor-UDCA) or pharmaceutically acceptable salt or ester thereof and at least one pharmaceutically acceptable excipient.

In embodiments of the invention, the present invention provides a tablet dosage form (immediate release tablet) comprising 24-nor-ursodeoxycholic acid (nor-UDCA) or pharmaceutically acceptable salt or ester thereof and at least one pharmaceutically acceptable excipient.

In a preferred embodiment of the present invention, nor-UDCA is employed in crystalline form.

In a particularly preferred embodiment of the present invention, the crystalline nor-UDCA is employed in micronized form. The nor-UDCA in this case preferably has an average particle size D₅₀ of less than 50 μm, in particular between 1 and 20 μm.

In another embodiment of the invention, the present invention provides a tablet dosage form comprising at least about 30% w/w nor-UDCA or pharmaceutically acceptable salt or ester thereof and at least one pharmaceutically acceptable excipient.

In further embodiment of the invention, the present invention provides a pharmaceutical composition preferably tablet dosage form comprising (a) about 30% w/w to about 90% w/w nor-UDCA based on the total weight of the tablet and (b) at least one pharmaceutically acceptable excipient.

In another embodiment of the invention, the present invention provides a tablet dosage form comprising
(a) about 30% w/w to about 90% w/w nor-UDCA,
(b) about 5% w/w to about 30% w/w diluent,
(c) about 5% w/w to about 10% w/w binder,
(d) about 1% w/w to about 10% w/w disintegrant,
(e) about 0.2% w/w to about 2% w/w lubricant based on the total weight of the tablet and
(f) optionally the tablet is coated with a film coating composition.

In one specific embodiment of the invention, the present invention provides a tablet dosage form comprising
(a) about 30% w/w to about 90% w/w nor-UDCA and
(b) croscarmellose sodium as a disintegrating agent.

In one embodiment of the invention, the present invention provides a tablet dosage form comprising nor-UDCA as the active ingredient, microcrystalline cellulose, polyvinyl pyrrolidone, croscarmellose sodium and magnesium stearate; wherein about 10% w/w to about 20% w/w microcrystalline cellulose based on total amount of microcrystalline cellulose in the tablet is present in intra-granular portion and about 80% w/w to about 90% w/w microcrystalline cellulose based on total amount of microcrystalline cellulose in the tablet is present in the extra-granular portion and wherein whole amount of croscarmellose sodium is present in the extragranular portion.

In a specific embodiment of the invention, the present invention provides a tablet dosage form comprising
(a) about 30% w/w to about 90% w/w nor-UDCA,
(b) about 5% w/w to about 30% w/w microcrystalline cellulose,
(c) about 5% w/w to about 10% w/w polyvinyl pyrrolidone,
(d) about 1% w/w to about 10% w/w croscarmellose sodium and
(e) about 0.2% w/w to about 2% w/w magnesium stearate based on the total weight of the tablet.

In a more specific embodiment of the invention, the present invention provides a tablet dosage form comprising
(a) about 30% w/w to about 90% w/w nor-UDCA,
(b) about 5% w/w to about 30% w/w microcrystalline cellulose,
(c) about 5% w/w to about 10% w/w polyvinyl pyrrolidone,
(d) about 1% w/w to about 10% w/w croscarmellose sodium,
(e) about 0.2% w/w to about 2% w/w magnesium stearate based on the total weight of the tablet,
   wherein about 10% w/w to about 20% w/w microcrystalline cellulose based on the total amount of the microcrystalline cellulose is present in intra-granular portion and about 80% w/w to about 90% w/w microcrystalline cellulose is present in the extra-granular portion based on the total amount of microcrystalline cellulose, and wherein whole amount of croscarmellose sodium is present in the extragranular portion.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

The term “about” when used before a numerical value indicates that the value may vary within a reasonable range, such as within +10%, +5% or =1 of the stated value.

As used herein, the term “comprising” or its grammatic variants is intended to mean that the compositions and methods, etc., include the recited elements, but do not exclude others.

As used herein, the term “comprising” or its grammatic variants is intended to mean that the compositions and methods, etc., include the recited elements, but do not exclude others.

“Consisting essentially of” or its grammatic variants when used to define compositions and methods, shall mean excluding other elements of any essential significance to the combination for the intended use, but not excluding elements that do not materially affect the characteristic(s) of the compositions or methods.

“Consisting of” or its grammatic variants shall mean excluding elements not specifically recited.

Embodiments defined by each of these transition terms are within the scope of this invention. For example, when a formulation is described as comprising ingredients A, B and C, a formulation consisting essentially of A, B and C, and a formulation consisting of A, B and C are independently within the scope of this invention.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

The term “wet granulation” or “moist granulation” as used herein, refers to the formation of granules using a granulation liquid (water, organic solvent, or a solution).

The term “dry granulation” as used herein, refers to the formation of granules without using a granulation liquid (water, organic solvent, or a solution).

The term “high-load solid tablet formulation” as used herein, refers to a solid tablet formulation comprising at least 30% w/w of nor-UDCA per tablet.

### DETAILED DESCRIPTION OF THE INVENTION

The present invention provides a pharmaceutical composition comprising 24-nor-ursodeoxycholic acid (nor-UDCA) or pharmaceutically acceptable salt or ester thereof and at least one pharmaceutically acceptable excipient.

In one embodiment of the invention the pharmaceutical composition is a solid dosage form for oral administration that includes tablets (immediate release tablets), effervescent or chewable tablets, orally disintegrating tablets, extended release tablets, capsules, extended release capsules, delayed release tablets, delayed release capsules, pills, powders and granules. Most preferably, the solid dosage form used in the present invention is tablet dosage form (immediate release tablet).

In embodiments of the invention, the present invention provides a tablet dosage form (immediate release tablet) comprising 24-nor-ursodeoxycholic acid (nor-UDCA) or pharmaceutically acceptable salt or ester thereof and at least one pharmaceutically acceptable excipient.

In one embodiment of the invention, the present invention provides a tablet dosage form (immediate release tablet) comprising nor-UDCA wherein the immediate release tablet having high bioavailability through improved dissolution, and a method for preparing it. The invention more particularly relates to an immediate release tablet for administration by oral route, containing nor-UDCA having poor aqueous solubility.

Nor-UDCA suffer from the disadvantage of being poorly soluble in an aqueous medium, thus having an insufficient dissolution profile and, consequently, poor bioavailability within the organism, following oral administration. The therapeutic dose required to be administered must thus be increased in order to obviate this disadvantage.

To improve the dissolution profile of nor-UDCA and its bioavailability, it would be useful to increase its dissolution so that it could attain a level close to 100%.

In a preferred embodiment of the present invention, nor-UDCA is employed in crystalline form.

In a particularly preferred embodiment of the present invention, the crystalline nor-UDCA is employed in micronized form. The nor-UDCA in this case preferably has an average particle size D₅₀ of less than 50 μm, in particular between 1 and 20 μm.

In embodiments of the invention, the present invention provides a tablet dosage form comprising at least about 30% w/w nor-UDCA or pharmaceutically acceptable salt or ester thereof and at least one pharmaceutically acceptable excipient.

In embodiments of the present invention, the present invention provides a tablet dosage form comprising at least about 30% w/w nor-UDCA or pharmaceutically acceptable salt or ester thereof, based on the total weight of the tablet dosage form, preferably in an amount of about 30% w/w to about 90% w/w nor-UDCA, more preferably of about 45% w/w to about 85% w/w nor-UDCA and most preferably of about 50% w/w to about 80% w/w nor-UDCA based on the total weight of the tablet dosage form.

In embodiments of the invention, the tablets according to the present invention allows for a higher drug load than those of the prior art, which exemplifies only tablets with the nor-UDCA (active ingredient) load of at least or greater than about 30% w/w. This higher nor-UDCA load allows for the preparation of smaller and lighter tablets that can be more easily swallowed. Moreover, a higher nor-UDCA load allows for higher dosages in a single tablet, such as 500 mg, 750 mg, 1000 mg and 1500 mg. This reduces the pill burden for the patients and thus increases patient compliance.

Further in another embodiment the high-load solid immediate release tablet of the present invention comprising at least or greater than about 30% w/w of nor-UDCA has low friability and good tensile strength.

In some embodiments, the pharmaceutical compositions described herein are prepared by a process comprising a wet granulation method.

In another embodiment is a solid tablet formulation comprising nor-UDCA, wherein the solid tablet formulation comprises at least about or greater than 30% w/w of nor-UDCA.

In another embodiment is a solid tablet formulation comprising nor-UDCA, wherein the solid tablet formulation comprises about 30% w/w to about 90% w/w of nor-UDCA.

In another embodiment is a high-load solid tablet formulation comprising at least 30% w/w of nor-UDCA, and one or more pharmaceutically acceptable excipients.

In another embodiment is a high-load solid tablet formulation comprising at least 40% w/w of nor-UDCA, and one or more pharmaceutically acceptable excipients.

In another embodiment is a high-load solid tablet formulation comprising at least 50% w/w of nor-UDCA, and one or more pharmaceutically acceptable excipients.

In another embodiment is a high-load solid tablet formulation comprising about 30% w/w to about 90% w/w of nor-UDCA, and one or more pharmaceutically acceptable excipients.

In another embodiment is a high-load solid tablet formulation comprising about 40% w/w to about 90% w/w of nor-UDCA, and one or more pharmaceutically acceptable excipients.

In another embodiment is a high-load solid tablet formulation comprising about 50% w/w to about 90% w/w of nor-UDCA, and one or more pharmaceutically acceptable excipients.

In another embodiment is a high-load solid tablet formulation comprising about 40% w/w to about 80% w/w of nor-UDCA, and one or more pharmaceutically acceptable excipients.

In another embodiment is a high-load solid tablet formulation comprising about 50% w/w to about 80% w/w of nor-UDCA and one or more pharmaceutically acceptable excipients.

In another embodiment is a high-load solid tablet formulation comprising about 60% w/w to about 80% w/w of nor-UDCA, and one or more pharmaceutically acceptable excipients.

In another embodiment is a high-load solid tablet formulation comprising about 50% w/w to about 80% w/w of nor-UDCA, and one or more pharmaceutically acceptable excipients.

In another embodiment is a high-load solid tablet formulation comprising about 50% w/w to about 80% w/w of nor-UDCA, and one or more pharmaceutically acceptable excipients, wherein the high-load solid tablet contains about 500 mg to about 1000 mg of nor-UDCA.

In another embodiment is a high-load solid tablet formulation comprising about 60% w/w to about 75% w/w of nor-UDCA, and one or more pharmaceutically acceptable excipients.

In another embodiment is a high-load solid tablet formulation comprising about 75% w/w to about 90% w/w of nor-UDCA, and one or more pharmaceutically acceptable excipients.

In a further embodiment is a high-load solid tablet formulation comprising about 75% w/w to about 90% w/w of nor-UDCA, wherein the high-load solid tablet contains greater than about 1000 mg to about 1500 mg of nor-UDCA.

In another embodiment is a high-load solid tablet formulation comprising at least 30% w/w of nor-UDCA, and one or more pharmaceutically acceptable excipients wherein the one or more excipients are present in an amount from about 10% w/w to about 70% w/w.

In another embodiment is a high-load solid tablet formulation comprising about 30% w/w to about 90% w/w of nor-UDCA, and one or more pharmaceutically acceptable excipients wherein the one or more excipients are present in an amount from about 10% w/w to about 70% w/w.

In another embodiment is a high-load solid tablet formulation comprising about 50% w/w to about 80% w/w of nor-UDCA, and one or more pharmaceutically acceptable excipients wherein the one or more excipients are present in an amount from about 20% w/w to about 50% w/w.

In another embodiment is a high-load solid tablet formulation comprising about 60% w/w to about 75% w/w of nor-UDCA, and one or more pharmaceutically acceptable excipients wherein the one or more excipients are present in an amount from about 25% w/w to about 40% w/w.

In further embodiment of the invention, the present invention provides a tablet dosage form comprising at least one pharmaceutically acceptable excipient selected from the group consisting of diluents, binders, disintegrants, lubricants and optionally coating agents for coating of the tablet dosage form.

In one embodiment of the invention the present invention provides a pharmaceutical composition comprising
(a) about 30% w/w to about 90% w/w nor-UDCA or a pharmaceutically acceptable salt thereof and
(b) one or more pharmaceutically acceptable excipients.

In another embodiment of the invention the present invention provides a pharmaceutical composition consisting essentially of
(a) about 30% w/w to about 90% w/w nor-UDCA or a pharmaceutically acceptable salt thereof and
(b) one or more pharmaceutically acceptable excipients.

In a further embodiment of the invention the present invention provides a pharmaceutical composition consisting of
(a) about 30% w/w to about 90% w/w nor-UDCA or a pharmaceutically acceptable salt thereof and
(b) one or more pharmaceutically acceptable excipients.

In another embodiment of the invention the present invention provides a high-load solid tablet comprising
(a) about 30% w/w to about 90% w/w nor-UDCA or a pharmaceutically acceptable salt thereof and
(b) one or more pharmaceutically acceptable excipients.

In further embodiment of the invention the present invention provides a high-load solid tablet consisting essentially of
(a) about 30% w/w to about 90% w/w nor-UDCA or a pharmaceutically acceptable salt thereof and
(b) one or more pharmaceutically acceptable excipients.

In embodiments of the invention the present invention provides a pharmaceutical composition comprising
(a) about 30% w/w to about 90% w/w nor-UDCA or a pharmaceutically acceptable salt thereof and
(b) one or more pharmaceutically acceptable excipient selected from group comprising diluents, binders, disintegrants, lubricants and film coating agents.

In embodiments of the invention the present invention provides a pharmaceutical composition consisting essentially of
(a) about 30% w/w to about 90% w/w nor-UDCA or a pharmaceutically acceptable salt thereof and
(b) one or more pharmaceutically acceptable excipient selected from group comprising diluents, binders, disintegrants, lubricants and film coating agents.

In another embodiment of the invention the present invention provides a pharmaceutical composition consisting of
(a) about 30% w/w to about 90% w/w nor-UDCA or a pharmaceutically acceptable salt thereof and
(b) one or more pharmaceutically acceptable excipient selected from group comprising diluents, binders, disintegrants, lubricants and film coating agents.

In one embodiment of the invention, the immediate release tablet contains about 100 mg to about 2000 mg of nor-UDCA, preferably each immediate release tablet contains 100 mg, 200 mg, 250 mg, 300 mg, 500 mg, 750 mg, 1000 mg, 1500 mg, 1750 mg and 2000 mg of nor-UDCA, more preferably about 500 mg, 750 mg, 1000 mg and 1500 mg of nor-UDCA.

In embodiments of the invention, diluents used in the present invention are selected from the group consisting of microcrystalline cellulose, silicified microcrystalline cellulose, starch, dicalcium phosphate, tricalcium phosphate, magnesium trisilicate, mannitol, maltitol, sorbitol, xylitol, lactose (anhydrous or as a hydrate, for example monohydrate), dextrose, maltose, sucrose, glucose, fructose, or maltodextrins. In preferred embodiment, diluent is present in an amount of about 1% w/w to about 50% w/w, preferably about 2% w/w to about 45% w/w, more preferably about 3% w/w to about 40% w/w and most preferably about 5% w/w to about 30% w/w based on the total weight of the tablet dosage form. The most preferably used diluent in the present tablet dosage form is microcrystalline cellulose.

In embodiments of the invention, tablet dosage form of the present invention contains microcrystalline cellulose as a diluent in amount of 1% w/w to about 50% w/w, preferably about 2% w/w to about 45% w/w, more preferably about 3% w/w to about 40% w/w and most preferably about 5% w/w to about 30% w/w based on the total weight of the tablet dosage form.

In further specific embodiment of the invention, the tablet dosage form of the present invention contains microcrystalline cellulose in amount of about 10% w/w to about 20% w/w based on the total amount of microcrystalline cellulose in the tablet in intra-granular portion and about 80% w/w to about 90% w/w microcrystalline cellulose based on total amount of microcrystalline cellulose in the tablet in the extra-granular portion.

In further embodiments of invention, tablet dosage form of the present invention contains a binder selected from group consisting of polyvinyl pyrrolidone, hydroxypropyl methylcellulose, hydroxypropyl cellulose including any mixtures thereof. In preferred embodiment, binder is present in an amount of about 1% w/w to about 25% w/w, preferably about 2% w/w to about 20% w/w, more preferably about 3% w/w to about 15% w/w and most preferably about 5% w/w to about 10% w/w based on the total weight of the tablet dosage form.

In preferred embodiments of the invention, tablet dosage form of present invention contains polyvinyl pyrrolidone as a binder and is most preferably used in the amount of about 5% w/w to about 10% w/w based on the total weight of the tablet dosage form.

In embodiments of the invention, tablet dosage form of the present invention contains disintegrants selected from the group consisting of sodium starch glycolate, crospovidone and croscarmellose sodium. In preferred embodiment, the tablet dosage form of the present invention contains of about 0.2% w/w to about 20% w/w of disintegrant based on the total weight of the tablet dosage form and most preferably of about 1% w/w to about 10% w/w of disintegrant based on the total weight of the tablet dosage form.

In one embodiment of the invention, the disintegration time of high-load solid immediate release tablet of the present invention is less than about 20 minutes, preferably less than about 15 minutes, more preferably less than about 10 minutes, even more preferably less than about 7.5 minutes and most preferably less than 5 minutes.

The inventors of the present invention have surprisingly found that the disintegration time of the high-load immediate release tablet comprising greater than about 30% w/w to about 90% w/w of nor-UDCA has less disintegration time of less than about 20 minutes, wherein the tablet dosage form contains of about 500 mg, 750 mg, 1000 mg, 1500 mg and 2000 mg of nor-UDCA.

The inventors of the present invention have surprisingly found that the dissolution or release of nor-UDCA from the high-load immediate release tablet comprising greater than about 30% w/w to about 90% w/w of nor-UDCA has dissolution or release of greater than 75% in about 30 minutes, wherein the tablet dosage form contains of about 500 mg, 750 mg, 1000 mg, 1500 mg and 2000 mg of nor-UDCA.

In another embodiment of the invention, the dissolution or release of nor-UDCA from the high-load solid immediate release tablet of the present invention has dissolution or release of greater than 75% in about 30 minutes.

In another embodiment of the invention, the dissolution or release of nor-UDCA from the high-load solid immediate release tablets is close to 100% (or, in any case, better than the following limits: 10% in 5 minutes, 20% in 10 minutes, 50% in 20 minutes and 75% in 30 minutes in a medium consisting of 900 ml water to which 4% β-Cyclodextrin buffer is added, with a blade rotation speed of 75 rpm of USP Type-II apparatus.

In the most preferred embodiment, the tablet dosage form of the present invention most preferably contains croscarmellose sodium as the disintegrant. Surprisingly the present inventors have found that the croscarmellose sodium is most preferred disintegrant, as the tablet dosage with croscarmellose sodium has significant less disintegration time when compared to the tablet dosage form containing sodium starch glycolate and crospovidone as the disintegrant. Further the present inventors have surprisingly found that the disintegrating time of the tablet is very less when the whole amount of croscarmellose sodium is used in the extra-granular portion of the tablet, compared to the tablet containing whole amount of croscarmellose sodium in the intra-granular portion and the tablet containing the croscarmellose sodium in both the intra-granular and extra-granular portion. In most preferred embodiment, the tablet dosage form of the present invention contains of about 0.2% w/w to about 20% w/w of croscarmellose sodium as a disintegrant based on the total weight of the tablet dosage form and most preferably of about 1% w/w to about 10% w/w of croscarmellose sodium as a disintegrant based on the total weight of the tablet dosage form.

In embodiments of the invention, the present invention provides a tablet dosage form comprising
(a) about 30% w/w to about 90% w/w nor-UDCA and
(b) croscarmellose sodium as a disintegrating agent.

In embodiments of the invention, tablet dosage form of the present invention contains lubricant selected from the group consisting of magnesium stearate and sodium stearyl fumarate. The most preferably used lubricant in the present invention is magnesium stearate and is used in the present invention in an amount of about 0.2% w/w to about 2% w/w based on the total weight of the tablet dosage form.

In embodiments of the present invention, tablet dosage form is film coated with a coating material Opadry. A typical composition of coating material Opadry could be combination of Polyvinyl alcohol, Polyethylene glycol, Talc and Titanium dioxide.

In embodiments of the invention, the tablet dosage form of the present invention does not comprise a surfactant, preferably selected from sodium lauryl sulfate, polysorbate 80 and poloxamer.

In another embodiment of the invention, the present invention provides a tablet dosage form comprising
(a) about 30% w/w to about 90% w/w nor-UDCA,
(b) about 5% w/w to about 30% w/w diluent,
(c) about 5% w/w to about 10% w/w binder,
(d) about 1% w/w to about 10% w/w disintegrant,
(e) about 0.2% w/w to about 2% w/w lubricant based on the total weight of the tablet and
(f) optionally the tablet is coated with a film coating composition.

In the most preferred embodiment, the present inventors have surprisingly found that the disintegration time of the tablet dosage form is significant less about 5 minutes, and more preferably about 1 minute 50 seconds comprising the combination of microcrystalline cellulose as a diluent and the croscarmellose sodium as a disintegrant, wherein about 10% w/w to about 20% w/w of microcrystalline cellulose based on the total amount of microcrystalline cellulose in tablet is present in intra-granular portion and about 80% w/w to about 90% w/w microcrystalline cellulose based on total amount of microcrystalline cellulose is present in the extra-granular portion, and wherein whole amount of croscarmellose sodium is present in the extra-granular portion.

In embodiments of the invention, the present invention provides a tablet dosage form comprising nor-UDCA as the active ingredient, microcrystalline cellulose, polyvinyl pyrrolidone, croscarmellose sodium and magnesium stearate, wherein whole amount of the croscarmellose sodium is present in the extra-granular portion of the tablet.

In one embodiment of the invention, the present invention provides a tablet dosage form comprising nor-UDCA as the active ingredient, microcrystalline cellulose, polyvinyl pyrrolidone, croscarmellose sodium and magnesium stearate; wherein about 10% w/w to about 20% w/w microcrystalline cellulose is present in intra-granular portion based on the total amount of the microcrystalline cellulose and about 80% w/w to about 90% w/w microcrystalline cellulose based on total amount of microcrystalline cellulose is present in the extra-granular portion, and wherein whole amount of croscarmellose sodium is present in the extra-granular portion.

In a specific embodiment of the invention, the present invention provides a tablet dosage form comprising
(a) about 30% w/w to about 90% w/w nor-UDCA,
(b) about 5% w/w to about 30% w/w microcrystalline cellulose,
(c) about 5% w/w to about 10% w/w polyvinyl pyrrolidone,
(d) about 1% w/w to about 10% w/w croscarmellose sodium and
(e) about 0.2% w/w to about 2% w/w magnesium stearate based on the total weight of the tablet.

In a more specific embodiment of the invention, the present invention provides a tablet dosage form comprising
(a) about 30% w/w to about 90% w/w nor-UDCA,
(b) about 5% w/w to about 30% w/w microcrystalline cellulose,
(c) about 5% w/w to about 10% w/w polyvinyl pyrrolidone,
(d) about 1% w/w to about 10% w/w croscarmellose sodium,
(e) about 0.2% w/w to about 2% w/w magnesium stearate based on the total weight of the tablet,
   wherein about 10% w/w to about 20% w/w microcrystalline cellulose based on the total amount of the microcrystalline cellulose is present in intra-granular portion and about 80% w/w to about 90% w/w total microcrystalline cellulose based on total amount of microcrystalline cellulose is present in the extra-granular portion, and wherein whole amount of croscarmellose sodium is present in the extra-granular portion.

In embodiments of the invention, the present invention relates to the process for the preparation of the tablet by wet/moist granulation method, with preferably rapid mixer granulation method or fluid-bed granulation method.

In another embodiment of the invention, the present invention relates to the process for the preparation of the tablet by dry granulation method, more preferably by roller compaction method.

In embodiments of the invention, the present invention provides the process for preparation of tablet comprising nor-UDCA in which,
(a) first granules containing nor-UDCA are prepared by the moist granulation and
(b) the granules are then converted to tablet dosage form with the addition of whole amount of croscarmellose sodium and other pharmaceutically acceptable excipients.

In embodiments of the invention, the present invention provides the process for preparation of tablet comprising nor-UDCA in which,
(a) first granules comprising the nor-UDCA, about 10% w/w to about 20% w/w microcrystalline cellulose based on total amount of microcrystalline cellulose and polyvinyl pyrrolidone are prepared by moist granulation; and
(b) the granules are then converted into the tablet dosage form with the addition of about 80% w/w to about 90% w/w total microcrystalline cellulose based on total amount of microcrystalline cellulose and whole amount of croscarmellose sodium and magnesium stearate.

In embodiments of the invention, the present invention relates to the tablet comprising nor-UDCA for the treatment of inflammatory cholestatic liver disease in a subject, wherein the inflammatory cholestatic liver disease is primary sclerosing cholangitis (PSC), primary biliary cirrhosis (PBC) or progressive familial intrahepatic cholestasis, in particular progressive familial intrahepatic cholestasis type 1, 2 and 3, cystic fibrosis, drug-induced cholestasis or a noncholestatic liver disease such as chronic viral hepatitis (B,C,D), alcoholic and non-alcoholic steatohepatitis, autoimmune hepatitis, hemochromatosis, Wilson disease and alpha-1-antitrypsin deficiency.

The following examples are provided to illustrate the present invention. It is understood, however, that the invention is not limited to the specific conditions or details described in the examples below. The examples should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### Example-1: Nor-UDCA Tablets Prepared by Direct Tableting without Granulation

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA | 500.00 | 83.33 |
| 2. | Silicified microcrystalline | 29.50 | 4.92 |
| | cellulose | | |
| 3. | Polyvinyl pyrrolidone | 30.00 | 5.00 |
| 4. | Sodium lauryl sulfate | 6.00 | 1.00 |
| 5. | Sodium starch glycolate | 30.00 | 5.00 |
| 6. | Magnesium Stearate | 4.50 | 0.75 |

| | | |
|---|---|---|
| TOTAL | 600.00 | 100.00 |

### Process for Preparation

1. Nor-UDCA, silicified microcrystalline cellulose, polyvinyl pyrrolidone, sodium lauryl sulfate and sodium starch glycolate are sifted and mixed to form a pre-lubrication blend.
2. The pre-lubrication blend was blended with magnesium stearate to form the lubricated blend.
3. The lubricated blend was direct compressed with suitable punches to form the tablet dosage form.

### Example-2: Nor-UDCA Tablets Prepared by Moist Granulation Process

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA (Micronized) | 500.00 | 83.33 |
| 2. | Microcrystalline cellulose | 17.50 | 2.92 |
| 3. | Polyvinyl pyrrolidone | 48.00 | 8.00 |
| 4. | Purified Water | q.s | q.s |

Extra-granular Portion

| | | | |
|---|---|---|---|
| 5. | Sodium starch glycolate | 30.00 | 5.00 |
| 6. | Magnesium Stearate | 4.50 | 0.75 |

| | | |
|---|---|---|
| TOTAL | 600.00 | 100.00 |

### Process for Preparation

1. Polyvinyl pyrrolidone was added slowly and dissolved in purified water to form the binder solution.
2. Nor-UDCA and microcrystalline cellulose was blended and mixed, further blended mixture was moist granulated with binder solution in rapid mixer granulator to form the moist granules.
3. Moist granules of step 2 are dried in fluid bed processor to form the dried granules.
4. Dried granules of step 3 was blended and mixed with sodium starch glycolate to form pre-lubricated mixture.
5. Pre-lubricated mixture of step 4 was blended and mixed with magnesium stearate to form the lubricated blend.
6. Lubricated blend of step 5 was compressed with suitable punches to form the tablet dosage form.

### Example-3: Nor-UDCA Tablets Prepared by Moist Granulation Process

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA (Micronized) | 500.00 | 83.33 |
| 2. | Microcrystalline cellulose | 17.50 | 2.92 |
| 3. | Sodium starch glycolate | 12.00 | 2.00 |
| 4. | Polyvinyl pyrrolidone | 42.00 | 7.00 |
| 5. | Sodium lauryl sulfate | 6.00 | 1.00 |
| 6. | Purified Water | q.s | q.s |

Extra-granular Portion

| | | | |
|---|---|---|---|
| 7. | Sodium starch glycolate | 18.00 | 3.00 |
| 8. | Magnesium Stearate | 4.50 | 0.75 |

| | | |
|---|---|---|
| TOTAL | 600.00 | 100.00 |

### Process for Preparation

1. Polyvinyl pyrrolidone and sodium lauryl sulfate was added slowly and dissolved in purified water to form the binder solution.
2. Nor-UDCA, microcrystalline cellulose, a portion of sodium starch glycolate, was blended and mixed, further blended mixture was moist granulated with binder solution in rapid mixer granulator to form the moist granules.
3. Moist granules of step 2 are dried in fluid bed processor to form the dried granules.
4. Dried granules of step 3 was blended and mixed with remaining portion of sodium starch glycolate to form pre-lubricated mixture.
5. Pre-lubricated mixture of step 4 was blended and mixed with magnesium stearate to form the lubricated blend.
6. Lubricated blend of step 5 was compressed with suitable punches to form the tablet dosage form.

### Example-4: Nor-UDCA Tablets Prepared by Moist Granulation Process

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA (Micronized) | 500.00 | 83.33 |
| 2. | Microcrystalline cellulose | 17.50 | 2.92 |
| 3. | Polyvinyl pyrrolidone | 42.00 | 7.00 |
| 4. | Sodium lauryl sulfate | 6.00 | 1.00 |
| 5. | Purified Water | q.s | q.s |

Extra-granular Portion

| | | | |
|---|---|---|---|
| 6. | Crospovidone | 30.00 | 5.00 |
| 7. | Magnesium Stearate | 4.50 | 0.75 |

| | | |
|---|---|---|
| TOTAL | 600.00 | 100.00 |

### Process for Preparation

1. Polyvinyl pyrrolidone and sodium lauryl sulfate was added slowly and dissolved in purified water to form the binder solution.
2. Nor-UDCA, microcrystalline cellulose was blended and mixed, further blended mixture was moist granulated with binder solution in rapid mixer granulator to form the moist granules.
3. Moist granules of step 2 are dried in fluid bed processor to form the dried granules.
4. Dried granules of step 3 was blended and mixed with crospovidone to form pre-lubricated mixture.
5. Pre-lubricated mixture of step 4 was blended and mixed with magnesium stearate to form the lubricated blend.
6. Lubricated blend of step 5 was compressed with suitable punches to form the tablet dosage form.

### Example-5: Nor-UDCA Tablets Prepared by Moist Granulation Process

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA (Micronized) | 500.00 | 83.33 |
| 2. | Microcrystalline cellulose | 17.50 | 2.92 |
| 3. | Polyvinyl pyrrolidone | 48.00 | 8.00 |
| 4. | Croscarmellose sodium | 15.00 | 2.50 |
| 5. | Purified Water | q.s | q.s |

Extra-granular Portion

| | | | |
|---|---|---|---|
| 6. | Croscarmellose sodium | 15.00 | 2.50 |
| 7. | Magnesium Stearate | 4.50 | 0.75 |

| | | |
|---|---|---|
| TOTAL | 600.00 | 100.00 |

### Process for Preparation

1. Polyvinyl pyrrolidone was added slowly and dissolved in purified water to form the binder solution.
2. Nor-UDCA, microcrystalline cellulose, and a portion of croscarmellose was blended and mixed, further blended mixture was moist granulated with binder solution in rapid mixer granulator to form the moist granules.
3. Moist granules of step 2 are dried in fluid bed processor to form the dried granules.
4. Dried granules of step 3 was blended and mixed with remaining portion of croscarmellose sodium to form pre-lubricated mixture.
5. Pre-lubricated mixture of step 4 was blended and mixed with magnesium stearate to form the lubricated blend.
6. Lubricated blend of step 5 was compressed with suitable punches to form the tablet dosage form.

### Example-6: Nor-UDCA Tablets Prepared by Moist Granulation Process

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA (Micronized) | 500.00 | 76.92 |
| 2. | Microcrystalline cellulose | 31.75 | 4.88 |
| 3. | Polyvinyl pyrrolidone | 32.50 | 5.00 |
| 4. | Croscarmellose sodium | 32.50 | 5.00 |
| 5. | Purified Water | q.s | q.s |

Extra-granular Portion

| | | | |
|---|---|---|---|
| 6. | Croscarmellose sodium | 48.75 | 7.50 |
| 7. | Magnesium Stearate | 4.50 | 0.69 |

| | | |
|---|---|---|
| TOTAL | 650.00 | 100.00 |

### Process for Preparation

1. Polyvinyl pyrrolidone was added slowly and dissolved in purified water to form the binder solution.
2. Nor-UDCA, microcrystalline cellulose, and a portion of croscarmellose was blended and mixed, further blended mixture was moist granulated with binder solution in rapid mixer granulator to form the moist granules.
3. Moist granules of step 2 are dried in fluid bed processor to form the dried granules.
4. Dried granules of step 3 was blended and mixed with remaining portion of croscarmellose sodium to form pre-lubricated mixture.
5. Pre-lubricated mixture of step 4 was blended and mixed with magnesium stearate to form the lubricated blend.
6. Lubricated blend of step 5 was compressed with suitable punches to form the tablet dosage form.

### Example-7: Nor-UDCA Tablets Prepared by Moist Granulation Process

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA (Micronized) | 500.00 | 71.43 |
| 2. | Microcrystalline cellulose | 18.98 | 2.7 |
| 3. | Polyvinyl pyrrolidone | 52.00 | 7.43 |
| 4. | Purified Water | q.s | q.s |

Extra-granular Portion

| | | | |
|---|---|---|---|
| 5. | Microcrystalline cellulose | 87.02 | 12.43 |
| 6. | Croscarmellose sodium | 35.00 | 5.00 |
| 7. | Magnesium Stearate | 7.00 | 1.00 |

| | | |
|---|---|---|
| TOTAL | 700.00 | 100.00 |

### Process for Preparation

1. Polyvinyl pyrrolidone was added slowly and dissolved in purified water to form the binder solution.
2. Nor-UDCA and a portion of microcrystalline cellulose was blended and mixed, further blended mixture was moist granulated with binder solution in rapid mixer granulator to form the moist granules.
3. Moist granules of step 2 are dried in fluid bed processor to form the dried granules.
4. Dried granules of step 3 was blended and mixed with croscarmellose sodium and remaining portion of microcrystalline cellulose to form pre-lubricated mixture.
5. Pre-lubricated mixture of step 4 was blended and mixed with magnesium stearate to form the lubricated blend.
6. Lubricated blend of step 5 was compressed with suitable punches to form the tablet dosage form.

### Example-8: Film Coating of Nor-UDCA of Example-7

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA (Micronized) | 500.00 | 68.68 |
| 2. | Microcrystalline cellulose | 18.98 | 2.61 |
| 3. | Polyvinyl pyrrolidone | 52.00 | 7.14 |
| 4. | Purified Water | q.s | q.s |

Extra-granular Portion

| 5. | Microcrystalline cellulose | 87.02 | 11.95 |
|---|---|---|---|
| 6. | Croscarmellose sodium | 35.00 | 4.81 |
| 7. | Magnesium Stearate | 7.00 | 0.96 |
| TOTAL (Core Tablet) | | 700.00 | |
| Coating Composition (15% w/w Dispersion) | | | |
| 8. | Opadry II white | 28.00 | 3.85 |
| 9. | Purified water | q.s | q.s |

| | | |
|---|---|---|
| Total weight of the Coated Tablet | 728.00 | 100.00 |

### Process for Preparation

1. The core tablet of nor-UDCA is prepared by the process as disclosed in Example-7.
2. The core tablets of example-7 are coated with Opadry coating dispersion prepared by dispersing Opadry II white material in purified water.

### Example-9: Disintegration Time Tablet Testing of Examples 1 to 8

The disintegration time of tablets in examples 1 to 8 are performed by using the Apparatus as disclosed in USP General Chapter 701 and the disintegration time of tablets are represented in following Table-1.

TABLE 1

| Example No | Disintegrating Time |
|---|---|
| Example-1 | Compactability of blend |
| | was poor to compress |
| | the tablets |
| Example-2 | 15 min 30 sec |
| Example-3 | 17 min 30 sec |
| Example-4 | 18 min 50 sec |
| Example-5 | 11 min 50 sec |
| Example-6 | 6 min 10 sec |
| Example-7 | 1 min 50 sec |
| Example-8 | 3 min 30 sec |

### Example-10: Dissolution Profile of Nor-UDCA Tablets of Example-8

Dissolution of tablets of example-8 are performed with USP Type-II (Paddle) Apparatus in purified water with 4% β-Cyclodextrin buffer/900 mL at 75 RPM of temperature 37±0.5° C. The dissolution results are represented in Table-2.

TABLE 2

| Time | % of nor- |
|---|---|
| points | UDCA |
| (mins) | Dissolved |
| 0 | 0.0 |
| 5 | 63.1 |
| 10 | 95.5 |
| 15 | 99.2 |
| 30 | 102.0 |
| 45 | 103.0 |
| 60 | 103.2 |

### Example-11: Nor-UDCA Tablets Prepared by Moist Granulation

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA (Micronized) | 500.00 | 76.92 |
| 2. | Microcrystalline cellulose | 32.50 | 5.00 |
| 3. | Polyvinyl pyrrolidone | 48.00 | 7.38 |
| 4. | Croscarmellose sodium | 32.50 | 5.00 |
| 5. | Purified Water | q.s | q.s |

Extra-granular Portion

| | | | |
|---|---|---|---|
| 6. | Croscarmellose sodium | 32.50 | 5.00 |
| 7. | Magnesium Stearate | 4.50 | 0.69 |

| | | |
|---|---|---|
| TOTAL | 650.00 | 100.00 |

### Process for Preparation

1. Polyvinyl pyrrolidone was added slowly and dissolved in purified water to form the binder solution.
2. Nor-UDCA, microcrystalline cellulose and a portion of croscarmellose sodium was blended and mixed, further blended mixture was moist granulated with binder solution in rapid mixer granulator to form the moist granules.
3. Moist granules of step 2 are dried in fluid bed processor to form the dried granules.
4. Dried granules of step 3 was blended and mixed with a portion of croscarmellose sodium to form pre-lubricated mixture.
5. Pre-lubricated mixture of step 4 was blended and mixed with magnesium stearate to form the lubricated blend.
6. Lubricated blend of step 5 was compressed with suitable punches to form the tablet dosage form.

### Example-12: Nor-UDCA Tablets Prepared by Moist Granulation

### Tablet Composition

Tablet Composition

| | | | |
|---|---|---|---|
| S. No | Ingredients | mg/Tablet | % w/w |

Intra-granular Portion

| | | | |
|---|---|---|---|
| 1. | Nor-UDCA (Micronized) | 500.00 | 62.50 |
| 2. | Microcrystalline cellulose | 18.98 | 2.37 |
| 3. | Polyvinylpyrrolidone | 52.00 | 6.50 |
| 4. | Purified water | q.s | q.s |

Extra-granular Portion

| | | | |
|---|---|---|---|
| 5. | Microcrystalline cellulose | 181.02 | 22.63 |
| 6. | Croscarmellose sodium | 40.00 | 5.00 |
| 7. | Magnesium Stearate | 8.00 | 1.00 |

| | | |
|---|---|---|
| TOTAL | 800.00 | 100.00 |

### Process for Preparation

1. Polyvinyl pyrrolidone was added slowly and dissolved in purified water to form the binder solution.
2. Nor-UDCA and a portion of microcrystalline cellulose was blended and mixed, further blended mixture was moist granulated with binder solution in rapid mixer granulator to form the moist granules.
3. Moist granules of step 2 are dried in fluid bed processor to form the dried granules.
4. Dried granules of step 3 was blended and mixed with croscarmellose sodium and remaining portion of microcrystalline cellulose to form pre-lubricated mixture.
5. Pre-lubricated mixture of step 4 was blended and mixed with magnesium stearate to form the lubricated blend.
6. Lubricated blend of step 5 was compressed with suitable punches to form the tablet dosage form.

### Example-13: Disintegration Time Tablet Testing of Examples 11 and 12

The disintegration time of tablets in examples 11 and 12 are performed by using the Apparatus as disclosed in USP General Chapter 701 and the disintegration time of tablets are represented in following Table-3.

TABLE 3

| Example No | Disintegrating Time |
|---|---|
| Example-11 | 15 Min 16 Seconds |
| Example-12 | 1 Min 30 Seconds |

### Example 14 to 17: Nor-UDCA Tablets Prepared by Moist Granulation

### Tablet Composition

Tablet Composition

| | | Ex: 14 | | Ex: 15 | | Ex: 16 | | Ex: 17 | |
|---|---|---|---|---|---|---|---|---|---|
| | | mg/ | % | mg/ | % | mg/ | % | mg/ | % |
| S. No | Ingredients | Tab | w/w | Tab | w/W | Tab | w/w | Tab | w/w |

Intra-granular Portion

| | | | | | | | | | |
|---|---|---|---|---|---|---|---|---|---|
| 1. | Nor-UDCA (Micronized) | 500.0 | 26.3 | 500.0 | 28.9 | 500.0 | 36.1 | 500.0 | 39.68 |
| 2. | Microcrystalline cellulose | 850.0 | 61.4 | 750.0 | 43.3 | 500.0 | 36.1 | 500.0 | 39.68 |
| 3. | Polyvinyl pyrrolidone | 110.0 | 7.9 | 100.0 | 5.8 | 60.0 | 4.33 | 60.0 | 4.76 |
| 4. | Purified water | q.s | q.s | q.s | q.s | q.s | q.s | q.s | q.s |

Extra-granular Portion

| | | | | | | | | | |
|---|---|---|---|---|---|---|---|---|---|
| 5. | Microcrystalline cellulose | 400.0 | 28.9 | 350.0 | 20.2 | 300.0 | 21.67 | 193.0 | 15.32 |
| 6. | Croscarmellose sodium | 31.0 | 2.2 | 25.0 | 1.4 | 17.5 | 1.26 | 0 | 0 |
| 7. | Magnesium stearate | 9.0 | 0.7 | 8 | 0.5 | 7.0 | 0.51 | 7.0 | 0.56 |

| | | | | |
|---|---|---|---|---|
| TOTAL | 1900.0 | 1733.0 | 1384.0 | 1260.0 |

### Process for Preparation

1. Polyvinyl pyrrolidone was added slowly and dissolved in purified water to form the binder solution.
2. Nor-UDCA and a portion of microcrystalline cellulose was blended and mixed, further blended mixture was moist granulated with binder solution in rapid mixer granulator to form the moist granules.
3. Moist granules of step 2 are dried in fluid bed processor to form the dried granules.
4. Dried granules of step 3 was blended and mixed with croscarmellose sodium and remaining portion of microcrystalline cellulose wherever required to form pre-lubricated mixture.
5. Pre-lubricated mixture of step 4 was blended and mixed with magnesium stearate to form the lubricated blend.
6. Lubricated blend of step 5 was compressed with suitable punches to form the tablet dosage form.

Observations: The disintegration time of tablets in Examples 14 to 17 are performed by using the Apparatus as disclosed in USP General Chapter 701 and the disintegration time of tablets are represented in following Table-4.

TABLE 4

| Example No | Disintegrating Time |
|---|---|
| Example-14 | 25 Min 16 Seconds |
| Example-15 | 22 Min 30 Seconds |
| Example-16 | 1 Min 10 Seconds |
| Example-17 | 1 Min 40 Seconds |

### Example 18 to 21: Nor-UDCA Tablets Prepared by Moist Granulation

### Tablet Composition

Tablet Composition

| | | Ex: 18 | | Ex: 19 | | Ex: 20 | | Ex: 21 | |
|---|---|---|---|---|---|---|---|---|---|
| | | mg/ | % | mg/ | % | mg/ | % | mg/ | % |
| S. No | Ingredients | Tab | w/w | Tab | w/w | Tab | w/w | Tab | w/w |

Intra-granular Portion

| | | | | | | | | | |
|---|---|---|---|---|---|---|---|---|---|
| 1. | Nor-UDCA (Micronized) | 500.0 | 84.8 | 500.0 | 88.5 | 500 | 94.0 | 500.0 | 91.4 |
| 2. | Microcrystalline cellulose | 20.0 | 3.4 | 20.0 | 3.5 | 10 | 1.9 | 15.0 | 2.7 |
| 3. | Polyvinyl pyrrolidone | 40.0 | 6.8 | 40.0 | 7.1 | 5 | 0.9 | 10.0 | 1.8 |
| 4. | Purified water | q.s | q. s | q. s | q.s | q.s | q. s | q. s | q.s |

Extra-granular Portion

| | | | | | | | | | |
|---|---|---|---|---|---|---|---|---|---|
| 5. | Microcrystalline cellulose | 5.0 | 0.8 | 0.0 | 0.0 | 5.0 | 0.9 | 10.0 | 1.8 |
| 6. | Croscarmellose sodium | 17.5 | 3.0 | 0.0 | 0.0 | 5.0 | 0.9 | 5.0 | 0.9 |
| 7. | Magnesium Stearate | 7.0 | 1.2 | 5.0 | 0.9 | 7.0 | 1.3 | 7.0 | 1.3 |

| | | | | |
|---|---|---|---|---|
| TOTAL | 589.50 | 565.0 | 532.0 | 547.0 |

The process for the preparation was similar to the process for preparation as disclosed in examples 14-17.

Tablets with compositions of Examples 20 and 21 could not be compressed due to compactability issues.

Observations: The disintegration time of tablets in Examples 18 to 19 as performed by using the Apparatus as disclosed in USP General Chapter 701 and the disintegration time of tablets are represented in following Table-5.

TABLE 5

| Example No | Disintegrating Time |
|---|---|
| Example-18 | 2 Min 40 Seconds |
| Example-19 | 6 Min 20 Seconds |

### Example 22: Nor-UDCA Tablets Prepared by Direct Compression

### Tablet Composition

Tablet Composition

| | | Ex: 22 | |
|---|---|---|---|
| S. No | Ingredients | mg/Tab | % w/w |
| 1. | Nor-UDCA (Micronized) | 500.0 | 99.0 |
| 2. | Magnesium Stearate | 5 | 1.0 |

| | |
|---|---|
| TOTAL | 505 |

Observations: When Nor-UDCA of 99% w/w and 1.0% w/w of Magnesium Stearate was mixed and compressed by direct compression, compactability of blend was poor to compress the tablets.

### Example 23 to 25: Nor-UDCA Tablets Prepared by Moist Granulation

### Tablet Composition

Tablet Composition

| | | Ex: 23 | | Ex:24 | | Ex: 25 | |
|---|---|---|---|---|---|---|---|
| | | mg/ | % | mg/ | % | mg/ | % |
| S. No | Ingredients | Tab | w/w | Tab | w/w | Tab | w/w |

Intra-granular Portion

| | | | | | | | |
|---|---|---|---|---|---|---|---|
| 1. | Nor-UDCA (Micronized) | 750.0 | 71.4 | 750.0 | 79.9 | 750.0 | 83.3 |
| 2. | Microcrystalline cellulose | 28.0 | 2.7 | 17.0 | 1.8 | 17.0 | 1.9 |
| 3. | Polyvinyl pyrrolidone | 80.0 | 7.6 | 50.0 | 5.3 | 40.0 | 4.4 |
| 4. | Purified water | q.s | q.s | q.s | q.s | q.s | q.s |

Extra-granular Portion

| | | | | | | | |
|---|---|---|---|---|---|---|---|
| 5. | Microcrystalline cellulose | 130.0 | 12.4 | 80.0 | 8.5 | 60.0 | 6.7 |
| 6. | Croscarmellose sodium | 52.0 | 5.0 | 35.0 | 3.7 | 25.0 | 2.8 |
| 7. | Magnesium stearate | 10.0 | 1.0 | 7.0 | 0.7 | 8.0 | 0.9 |

| | | | |
|---|---|---|---|
| TOTAL | 1050.0 | 939.0 | 900.0 |

The process for the preparation was similar to the process for preparation as disclosed in examples 14-17.

Observations: The disintegration time of tablets in Examples 23 to 25 are performed by using the Apparatus as disclosed in USP General Chapter 701 and the disintegration time of tablets are represented in following Table-6.

TABLE 6

| Example No | Disintegrating Time |
|---|---|
| Example-23 | 2 Min 40 Seconds |
| Example-24 | 3 Min 20 Seconds |
| Example-25 | 3 Min 10 Seconds |

### Example 26 to 28: Nor-UDCA Tablets Prepared by Moist Granulation

### Tablet Composition

Tablet Composition

| | | Ex: 26 | | Ex: 27 | | Ex: 28 | |
|---|---|---|---|---|---|---|---|
| | | mg/ | % | mg/ | % | mg/ | % |
| S. No | Ingredients | Tab | w/w | Tab | w/w | Tab | w/w |

Intra-granular Portion

| | | | | | | | |
|---|---|---|---|---|---|---|---|
| 1. | Nor-UDCA (Micronized) | 1000 | 71.4 | 1000 | 77.8 | 1000 | 85.5 |
| 2. | Microcrystalline cellulose | 38 | 2.7 | 35 | 2.7 | 25 | 2.1 |
| 3. | Polyvinyl pyrrolidone | 104 | 7.4 | 90 | 7.0 | 46 | 3.9 |
| 4. | Purified water | q.s | q.s | q.s | q.s | q.s | q.s |

Extra-granular Portion

| | | | | | | | |
|---|---|---|---|---|---|---|---|
| 5. | Microcrystalline cellulose | 174 | 12.4 | 100 | 7.8 | 55 | 4.7 |
| 6. | Croscarmellose sodium | 70 | 5.0 | 50 | 3.9 | 35 | 3.0 |
| 7. | Magnesium stearate | 14 | 1.0 | 10 | 0.8 | 8 | 0.7 |

| | | | |
|---|---|---|---|
| TOTAL | 1400 | 1285 | 1169 |

The process for the preparation was similar to the process for preparation as disclosed in examples 14-17.

Observations: The disintegration time of tablets in Examples 26 to 28 are performed by using the Apparatus as disclosed in USP General Chapter 701 and the disintegration time of tablets are represented in following Table-7.

TABLE 7

| Example No | Disintegrating Time |
|---|---|
| Example-26 | 1 Min 50 Seconds |
| Example-27 | 4 Min 30 Seconds |
| Example-28 | 5 Min 25 Seconds |

### Example 29 to 31: Nor-UDCA Tablets Prepared by Moist Granulation

### Tablet Composition

Tablet Composition

| | | Ex: 29 | | Ex: 30 | | Ex: 31 | |
|---|---|---|---|---|---|---|---|
| | | mg/ | % | mg/ | % | mg/ | % |
| S. No | Ingredients | Tab | w/w | Tab | w/w | Tab | w/w |

Intra-granular Portion

| | | | | | | | |
|---|---|---|---|---|---|---|---|
| 1. | Nor-UDCA (Micronized) | 1500 | 83.3 | 1500 | 85.7 | 1500 | 81.1 |
| 2. | Microcrystalline cellulose | 150 | 8.3 | 100 | 5.7 | 135 | 7.3 |
| 3. | Polyvinyl pyrrolidone | 55 | 3.1 | 55 | 3.1 | 55 | 3.0 |
| 4. | Purified water | q.s | q.s | q.s | q.s | q.s | q.s |

Extra-granular Portion

| | | | | | | | |
|---|---|---|---|---|---|---|---|
| 5. | Microcrystalline cellulose | 35 | 1.9 | 35 | 2.0 | 100 | 5.4 |
| 6. | Croscarmellose sodium | 50 | 2.8 | 50 | 2.9 | 50 | 2.7 |
| 7. | Magnesium stearate | 10 | 0.6 | 10 | 0.6 | 10 | 0.5 |

| | | | |
|---|---|---|---|
| TOTAL | 1800 | 1750 | 1850 |

The process for the preparation was similar to the process for preparation as disclosed in examples 14-17.

Observations: The disintegration time of tablets in Examples 29 to 31 are performed by using the Apparatus as disclosed in USP General Chapter 701 and the disintegration time of tablets are represented in following Table-8.

TABLE 8

| Example No | Disintegrating Time |
|---|---|
| Example-29 | 2 Min 20 Seconds |
| Example-30 | 3 Min 40 Seconds |
| Example-31 | 4 Min 90 Seconds |

We claim:

1. A pharmaceutical composition comprising
(a) about 30% w/w to about 90% w/w nor-UDCA or a pharmaceutically acceptable salt thereof and
(b) one or more pharmaceutically acceptable excipients.

2. The pharmaceutical composition as claimed in claim 1 comprising one or more pharmaceutically acceptable excipients selected from group comprising diluents, binders, disintegrants, lubricants and film coating agents.

3. The pharmaceutical composition as claimed in claim 1, wherein the composition comprises of about 250 mg to about 2000 mg of nor-UDCA.

4. The pharmaceutical composition as claimed in claim 1, wherein average particle size (D₅₀) of nor-UDCA less than 50 μm.

5. The pharmaceutical composition as claimed in claim 1, wherein the pharmaceutical composition is selected from the group consisting of immediate release tablets, effervescent tablets, chewable tablets, orally disintegrating tablets, chewable tablets, capsules, pills, powders and granules.

6. The pharmaceutical composition as claimed in claim 4, wherein the pharmaceutical composition is a high-load solid immediate release tablet.

7. The pharmaceutical composition as claimed in claim 1, wherein the high-load solid immediate release tablet comprises of
(a) about 5% w/w to about 30% w/w diluent,
(b) about 5% w/w to about 10% w/w binder,
(c) about 1% w/w to about 10% w/w disintegrant, and
(d) about 0.2% w/w to about 2% w/w lubricant.

8. The pharmaceutical composition as claimed in claim 7, wherein the high-load solid immediate release tablet comprises of
(a) about 5% w/w to about 30% w/w microcrystalline cellulose,
(b) about 5% w/w to about 10% w/w polyvinyl pyrrolidone,
(c) about 1% w/w to about 10% w/w croscarmellose sodium and
(d) about 0.2% w/w to about 2% w/w magnesium stearate based on the total weight of the tablet.
